(19) United States
(12) Patent Application Publication
(10) Pub. No.: US 2006/0217633 A1
(43) Pub. Date: Sep. 28, 2006

(54) Method for measuring pressures deriving from leakage current

(76) Inventors: Raymond Glocker, Dudingen (CH); Haldun Ozdemir, Düdingen (CH)

Correspondence Address: NIXON & VANDERHYE, PC, 901 NORTH GLEBE ROAD, 11TH FLOOR, ARLINGTON, VA, 22203, US

(21) Appl. No.: 10/552,074

(22) PCT Filed: Apr. 8, 2003
(86) PCT No.: PCT/CH03/00227

§ 371 Date: Nov. 17, 2005

(51) Int. Cl. A61B 5/00; A61B 5/02

(52) U.S. Cl. 600/561; 600/486

(57) ABSTRACT

A method for performing pressure measurements by means of pressure profile sensors technique comprises the following steps: a) introducing into the mammal a catheter having at least a portion of its wall which is sufficiently flexible to be deflected by external pressure; b) introducing progressively into the catheter lumen an electrically conductive liquid substance while applying to it simultaneously an alternative current and mechanical oscillations; c) detecting by means of an electrode placed at the external surface of the subject the leakage current induced by the liquid substance traveling in the catheter; d) transferring the leakage current thus recorded to a converter suitable to convert the leakage parameters provided thereto into pressure values; and e) displaying the pressure values as such or as a function of the measurement location or period to afford corresponding pressure profiles. This method is useful for performing pressure, respectively pressure profiles measurements in various mammal body tracts or cavities. The invention also relates to a device using said method.

### FIELD OF THE INVENTION

The invention refers to medical investigation or diagnostic tools and methods applicable to mammals, more specifically to methods for measuring pressures, respectively pressure profiles in various body tracts or cavities.

### BACKGROUND OF THE INVENTION

Among various technologies available for measuring pressures within the frame of medical investigations or diagnostic methods the Pressure Profile Sensor Method has been developed recently (PPS-Method). U.S. Pat. No. 6,459,972 of Sep. 17, 2002 and the literature cited therein provide detailed information concerning the fundamentals of that technique and its potential uses—see also “Pressure Profile Sensing Systems” in Sensors and Actuators A 93 (2001), 52-56, Elsevier Science B.V.

The basis of that highly performing technique is linked to the deformation of the shape of a saline solution caused by an external pressure when applied to a catheter lumen in which the saline solution is located provided the walls of the catheter tubing in which the saline solution is filled in are thin enough to be deflected by the external pressure. As the saline solution is electrically conducting, the capacitance of the same will change when the shape is changed through the applied pressure along the axis of the tubing and over time, the most sensitive part of the saline solution column being the front of same. So variations of the pressure can be followed over time by appropriate capacitive measurements.

Capacitance measurements as referred to here above, however, necessitate the use of expensive and complex material especially that used for manufacturing catheters incorporating conductive means. Accuracy and sensitivity of current methods also need improvement.

Consequently the medical community is still looking for easier, more efficient, more precise and more reliable methods and, if ever possible, less painful for the patients. The present invention provides a very efficient solution which easily overcomes all the obstacles currently met.

### SUMMARY OF THE INVENTION

The invention provides first a method for performing pressure, respectively pressure profile measurements in mammals by means of the pressure profile sensor technique, making use of the leakage current induced into the mammal body when introducing therein a catheter filled with an electrically excited liquid substance.

The invention further provides using the above method for performing pressure, respectively pressure profile measurements in body tracts or cavities such as esophagus, stomach, intestine, urinary tract or bladder, or blood vessels.

The invention, finally, provides an apparatus suitable for performing the method defined in claim 1. The apparatus itself is defined in claim 13.

### DETAILED DESCRIPTION OF THE INVENTION

The invention refers firstly to a method for performing pressure, respectively pressure profile measurement in mammals, which comprises
a) introducing into the mammal a catheter having at least a portion of its wall which is sufficiently flexible to be deflected by external pressure;
b) introducing progressively into the catheter lumen an electrically conductive liquid substance while applying simultaneously to it alternative current and mechanical oscillations;
c) detecting by means of an electrode placed at the external surface of the subject the leakage current induced by the liquid substance traveling trough the catheter;
d) transferring the leakage current thus recorded to a converter suitable to convert the leakage current parameters provided thereto into pressure values; and
e) displaying the pressure values as such, or as a function of the measurement location or measurement period or both to afford corresponding pressure profiles.

The invention further refers to an apparatus useful for performing the above method. This apparatus comprises
a source of an electrically conductive liquid substance connected to an alternative current source;
peristaltic pumping means fitted directly to the source of liquid substance;
mechanical oscillation means connected downwards to peristaltic pumping means;
an electrode placed at the external surface of the subject for recording and then transferring the detected leakage current to the converter;
a converter suitable for deriving pressure values from the leakage current parameters which have been transferred thereto; and
means suitable to display pressure values as such, or as a function of the measurement location or measurement period or both.

Specific embodiments of said apparatus shall appear when relevant, in the description here below.

The alternative current which applies to the liquid substance is in fact a low voltage/high frequency current. “Low voltage” means a current voltage which is easily supported by the patient, namely not detrimental to sensitive body parts or organs, and not painful, but strong enough to provide electrical excitations which will be eventually detected by means such an external electrode. Typical voltage which applies according to the invention is comprised between about 500 mV and about 6 V, preferably between about 1 V and about 4 V.

The frequency of the low voltage current applied to the liquid substance has to be high enough to go above the insulation barrier provided by the wall of the catheter currently used in that domain, more specifically of the magnitude of about 60 kHz. The upper value of said frequency will be defined as not interfering with e.g. natural or artificial heart stimulation and also not causing any pain to the patient. Said upper value, moreover, must also be fully compatible with the machines used according to the PPS-Method. Typically, said frequency will be comprised between about 60 kHz and about 130 kHz, preferably between about 80 kHz and about 120 kHz.

Preferred conditions for such an alternative current are 2 V/100 kHz; they fulfill all the regulatory and technical requirements currently met worldwide. The source of such an alternative current will be selected from tools usually met in the prior known technology.

The basic concept of the present invention resides in the fact that the electrically excited liquid column is subjected to mechanical oscillations. Said mechanical oscillations have controlled amplitude and frequency, the amplitude being of about max. 4 mm, preferably of about 2 mm and the frequency of about max 15, preferably of about 10 Hz. These values have proved non-disturbing the homogeneity of the liquid column currently used for such measurements, and consequently compatible with the accuracy of pressure measurements which is required. Related detailed explanations can be found in e.g. EP 02 505 490.0 filed Jun. 14, 2002.

According to the invention the electrically conductive liquid substance is moving progressively, either continuously or stepwise, through the catheter. Continuous liquid introduction can occur for moving the frontline of the liquid column to a certain location whereas step-by-step progression occurs when measurements take place. Said progression is achieved by means of a peristaltic pump directly fitted to the liquid source—see also EP 02 505 490.0—.

The mechanical oscillation means are located downwards to the peristaltic pump and comprise e.g. a stepper motor which can be triggered fast or slow to generate high or low frequency oscillations while controlling simultaneously the amplitude of the oscillations—see also EP 02 505 490.0—.

According to the invention the catheter has a portion which is sufficiently flexible to be deflected by the pressure which will be exerted on it once introduced into the subject. Said portion is preferably a longitudinal strip of the catheter which is thinner than the remaining portion of the catheter wall. Such a construction is easily achievable using the polymer extrusion or co-extrusion techniques.

Whenever required the catheter can be conveniently reinforced internally by a suitable fiber web.

The catheter is most frequently a single lumen catheter. It can also be a multi-lumen catheter as that described in PCT/CH 03/0013 filed Feb. 17, 2003.

Said catheter is made of innocuous polymer plastic material, preferably of non-conductive polymer material and it does not need to comprise conductive material such as metal strips or inner metal coating. This allows manufacturing thin and flexible tubes which are cheaper and easier to handle than those used for the prior known technology—see e.g. U.S. Pat. No. 6,459,972. Such catheters revealed furthermore definitely less painful for the patients. Suitable polymer material can be selected among silicon, rubber, latex, polyurethane, PVC, polypropylene, PE or the like.

According to the invention the electrically conductive liquid substance is an aqueous liquid like a saline solution, e.g. a NaCl or KCl solution.

According to the invention the leakage current induced by the electrically excited saline solution progressing through the catheter is recorded by a currently available electrode placed at the external surface of the subject body. One of the main features of the present invention resides in the fact that the leakage current thus induced is strong and homogenous enough to reflect accurately the pressure values measured at a given location. When compared to prior known PPS-Method e.g. according to U.S. Pat. No. 6,459,972, which is based on capacitance measurements, the method according to the present invention proved definitely more sensitive and more precise for the same electrical excitation parameters.

Convenient recording of the leakage current is performed by means of one external electrode only, which can be fixed at any suitable body part. The leakage current is then transferred to a converter suitable to convert leakage current parameters into corresponding pressure values which can be displayed eventually in the form of graphs e.g. on paper strips or computer screens.

Pressure values may appear as such for a given location and at a given time. They can also be calculated for a given location but over a certain period and so display the pressure variation or evolution over time; they can be furthermore detected while the front line of the electrically excited liquid is progressing step-by-step through the catheter to afford corresponding pressure profiles. Further details of these types of measurements appear in EP 02 505 490.0.

According to the invention, eventually, the method can be used to for perform real time pressure, respectively pressure profile measurements. It can be used also to perform ex-temporaneum pressure, respectively pressure profiles measurements as well, by recording the pressure values provided by the converter and by displaying them at a time different from that of the leakage current recording.

The method described here above has proved highly efficient for performing pressure, respectively pressure profile measurements in mammal body tracts or cavities such as lung, esophagus, stomach, intestine, urinary tract or bladder, or blood vessels.

1. A method for performing pressure, respectively pressure profile, measurements in mammals by means of the pressure profile sensors technique, which comprises
a) introducing into the mammal a catheter having at least a portion of its wall which is sufficiently flexible to be deflected by external pressure;
b) introducing progressively into the catheter lumen an electrically conductive liquid substance while applying simultaneously to it alternative current and mechanical oscillations;
c) detecting by means of an electrode placed at the external surface of the subject the leakage current induced by the liquid substance traveling through the catheter;
d) transferring the leakage current thus recorded to a converter suitable to convert the leakage current parameters provided thereto into corresponding pressure values; and
e) displaying the pressure values as such, or as a function of the measurement location or measurement period or both to afford corresponding pressure profiles.

2. Method of claim 1, wherein the alternative current is a low voltage/high frequency current and wherein the mechanical oscillations have controlled amplitude and frequency.

3. Method according to claim 1, wherein the catheter is made of innocuous polymer plastic material, preferably of non-conductive innocuous polymer plastic material.

4. Method according to claim 1, wherein the catheter is a single lumen or a multi-lumen catheter.

5. Method according to claim 1, wherein the electrically conductive liquid substance is an aqueous liquid, preferably a saline solution.

6. Method according to claim 1, wherein the liquid substance is progressing step-by-step through the catheter lumen.

7. Method according claims 1, wherein the alternative current voltage applied to the liquid substance is comprised between about 500 mV and about 6 V, preferably between about 1 and about 4 V.

8. Method according to claim 1, wherein the alternative current frequency applied to the liquid substance is comprised between about 60 and 130 kHz, preferably between about 80 and 120 kHz.

9. Method according to claim 1, wherein the mechanical oscillations applied to the liquid substance have an amplitude of about max. 4 mm and a frequency of about max 15 Hz, preferably of about 2 mm, respectively about 10 Hz.

10. Use of the method according to claim 1 for performing pressure, respectively pressure profile measurements in mammal body tracts or cavities such as lung, esophagus, stomach, intestine, urinary tract or bladder, or blood vessels.

11. Use of the method according to claim 1 for performing real time pressure, respectively pressure profile measurements.

12. Use of the method of claim 1 for performing ex-temporaneum pressure, respectively pressure profiles measurements by recording the pressure values provided by the converter and by displaying them at a time different from that of the leakage current recording.

13. An apparatus for performing the method of claim 1, which comprises
a source of an electrically conductive liquid substance connected to an alternative current source;
peristaltic pumping means fitted directly to the source of liquid substance;
mechanical oscillation means connected downwards to peristaltic pumping means;
an electrode placed at the external surface of the subject for recording and then transferring the detected leakage current to the converter;
a converter suitable for deriving pressure values from the leakage current parameters which have been transferred thereto; and
means suitable to display pressure values as such, or as a function of the measurement location or measurement period or both.
